(19) United States
(12) Patent Application Publication
(10) Pub. No.: US 2024/0115429 A1
(43) Pub. Date: Apr. 11, 2024

(54) ABSORBENT ARTICLE

(71) Applicant: Kimberly-Clark Worldwide, Inc., Neenah, WI (US)

(72) Inventors: Benjamin P. Sierra, Appleton, WI (US); Nick L. Hooper, Appleton, WI (US); Jason K. Sieck, Neenah, WI (US); Brian R. Vogt, Larsen, WI (US); Rebecca C. Mohr, Appleton, WI (US)

(21) Appl. No.: 18/263,199

(22) PCT Filed: Feb. 16, 2021
(86) PCT No.: PCT/US21/18144

§ 371 Date: Jul. 27, 2023

(51) Int. Cl. A61F 13/475; A61F 13/472; A61F 13/476; A61F 13/511

(52) U.S. Cl. CPC A61F 13/4756; A61F 13/472; A61F 13/476; A61F 13/51104

(57) ABSTRACT

An absorbent article can have a target acquisition region which is the region of the absorbent article wherein initial acquisition of body exudates from the wearer of the absorbent article should occur. To identify the target acquisition region for the wearer of the absorbent article, the target acquisition region is defined by and bounded by a perimeter formed of individual embossment points. To provide the wearer of the absorbent article with the knowledge that the absorbent article will be able to capture and store their body exudates, the absorbent article has a second perimeter formed of individual embossment points positioned between the perimeter defining the target acquisition region and the edge of the absorbent core. A minimum distance between an embossment point of the first perimeter and an embossment point of the second perimeter is at least 6 mm.

### BACKGROUND

Products such as absorbent articles are often used to collect and retain human body exudates containing, for example, urine, menses and/or blood. Comfort, absorbency, and discretion are three main product attributes and areas of concern for the wearer of the product. In particular, a wearer is often interested in knowing that such products will absorb significant volumes of body exudates with minimal leakage in order to protect their undergarments, outer garments, or bedsheets from staining, and that such products will help them avoid the subsequent embarrassment brought on by such staining.

Currently, a wide variety of products for absorption of body exudates are available in the form of feminine pads, sanitary napkins, panty shields, pantiliners, and incontinence devices. These products generally have an absorbent core positioned between a body-facing liquid permeable topsheet layer and a garment-facing liquid impermeable backsheet layer. The edges of the topsheet and the backsheet layers are often bonded together at their periphery to form a seal to contain the absorbent core and body exudates received into the product through the topsheet layer. In use, such products are typically positioned in the crotch portion of an undergarment for absorption of the body exudates and a garment attachment adhesive on the backsheet layer can be used to attach the product to the inner crotch portion of the undergarment. Some of these products can also include wing-like structures for wrapping about the wearer's undergarment to further secure the product to the undergarment and to protect the undergarment from staining. Such wing-like structures (also known as flaps or tabs) are frequently made from lateral extensions of the topsheet and/or backsheet layers.

Many absorbent articles are provided with embossing patterns for aesthetic purposes and/or to communicate to the wearer that the absorbent article will perform as intended to capture and contain body exudates. One problem with such conventional absorbent articles is that the embossing patterns are too close to each other and can result in stiff, inflexible, non-compressible absorbent articles. Such deficiencies in an absorbent article can result in a product that is not comfortable for the wearer to use.

As a result, there remains a need for an absorbent article which incorporates embossing patterns in a manner in which they can communicate to the wearer the absorbent article will function as intended to capture and contain body exudates while not resulting in an absorbent article that is stiff, inflexible, and non-compressible.

### SUMMARY

In various embodiments, an absorbent article can have a topsheet layer and a backsheet layer; an absorbent core comprising an absorbent core edge wherein the absorbent core is positioned between the topsheet layer and the backsheet layer; a target acquisition region bounded by a first perimeter comprising a first plurality of embossment points; and a second perimeter comprising a second plurality of embossment points positioned between the first perimeter and the absorbent core edge; wherein a center of each of the first plurality of embossment points is separated by a first distance from a center of each of the second plurality of embossment points and the first distance is at least 6 mm. In various embodiments, the first distance is at least 10 mm.

In various embodiments, the first plurality of embossment points is positioned within a first embossment region. In various embodiments, the first embossment region has a pair of sidewalls separated from each other by a distance of 2 to 3.5 mm.

In various embodiments, the second plurality of embossment points is positioned within a second embossment region. In various embodiments, the second embossment region has a pair of sidewalls separated from each other by a distance of 2 to 3.5 mm.

In various embodiments, each of the first embossment points within the first plurality of embossment points are separated from each other by a second distance of from 0.7 mm to 3.5 mm. In various embodiments, each of the second embossment points within the plurality of second embossment points are separated from each other by a third distance of from 0.5 mm to 6.25 mm.

In various embodiments, the absorbent article can further have a fluid intake layer positioned between the absorbent core and the fluid intake layer.

In various embodiments, the absorbent article can further have a shaping element.

In various embodiments, the absorbent article can further have a pair of wings.

### BRIEF DESCRIPTION OF THE DRAWINGS

FIG. 1 is a top down view of an embodiment of an absorbent article.

FIG. 2 is a cross-sectional view of the absorbent article of FIG. 1 taken along line 2-2.

FIG. 3 is a cross-sectional view of the absorbent article of FIG. 1 taken along line 3-3.

FIG. 4 is a close-up view of a portion of the absorbent article of FIG. 1.

FIG. 5 is a partial view of a ridge on an embossing roller.

FIG. 6 is a top down view of an embodiment of an absorbent article.

FIG. 7 is a cross-sectional view of the absorbent article of FIG. 6 taken along line 7-7.

FIG. 8 is a cross-sectional view of the absorbent article of FIG. 6 taken along line 8-8.

FIG. 9 is a close-up view of a portion of the absorbent article of FIG. 7.

FIG. 10 is a top down view of an embodiment of an absorbent article.

FIG. 11 is a top down view of an embodiment of an absorbent article.

FIG. 12 is a top down view of an embodiment of an absorbent article without embossment points.

FIG. 13 is a chart showing side compression test results.

### DETAILED DESCRIPTION

The present disclosure is generally directed towards an absorbent article which has a target acquisition region which is the region of the absorbent article wherein initial acquisition of body exudates from the wearer of the absorbent article should occur. To identify the target acquisition region for the wearer of the absorbent article, the target acquisition region is defined by and bounded by a first perimeter formed of individual embossment points. To provide the wearer of the absorbent article with the knowledge that the absorbent article will be able to capture and store their body exudates, the absorbent article has a second perimeter formed of individual embossment points positioned between the perimeter defining the target acquisition region and the edge of the absorbent core. A minimum distance between the centers of the embossment points of the first perimeter and the centers of the embossment points of the second perimeter is at least 6 mm.

### Definitions

As used herein, the term “absorbent article” refers herein to a garment or other end-use personal care absorbent article, including, but not limited to, catamenial products, such as sanitary napkins, feminine pads, pantiliners, and panty shields, incontinence devices, and the like.

As used herein, the term “airlaid” refers herein to a web manufactured by an airlaying process. In the airlaying process, bundles of small fibers having typical lengths ranging from about 3 to about 52 mm are separated and entrained in an air supply and then deposited onto a forming screen, usually with the assistance of a vacuum supply. The randomly deposited fibers are then bonded to one another using, for example, hot air to activate a binder component or a latex adhesive. Airlaying is taught in, for example, U.S. Pat. No. 4,640,810 to Laursen, et al., which is incorporated herein in its entirety by reference thereto for all purposes.

As used herein, the term “bonded” refers herein to the joining, adhering, connecting, attaching, or the like, of two elements. Two elements will be considered bonded together when they are joined, adhered, connected, attached, or the like, directly to one another or indirectly to one another, such as when bonded to an intermediate element. The bonding can occur via, for example, adhesive, pressure bonding, thermal bonding, ultrasonic bonding, stitching, suturing, and/or welding.

As used herein, the term “bonded carded web” refers herein to webs that are made from staple fibers which are sent through a combing or carding unit which separates or breaks apart and aligns the staple fibers in the machine direction to form a generally machine direction oriented fibrous nonwoven web. This material may be bonded together by methods that can include point bonding, through air bonding, ultrasonic bonding, adhesive bonding, etc.

As used herein, the term “coform” refers herein to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff, and also superabsorbent particles, inorganic and/or organic absorbent materials, treated polymeric staple fibers and so forth. Some examples of such coform materials are disclosed in U.S. Pat. No. 4,100,324 to Anderson, et al., U.S. Pat. No. 4,818,464 to Lau, U.S. Pat. No. 5,284,703 to Everhart, et al., and U.S. Pat. No. 5,350,624 to Georger, et al., each of which are incorporated herein in their entirety by reference thereto for all purposes.

As used herein, the term “conjugate fibers” refers herein to fibers which have been formed from at least two polymer sources extruded from separate extruders and spun together to form one fiber. Conjugate fibers are also sometimes referred to as bicomponent fibers or multicomponent fibers. The polymers are arranged in substantially constantly positioned distinct zones across the cross-sections of the conjugate fibers and extend continuously along the length of the conjugate fibers. The configuration of such a conjugate fiber may be, for example, a sheath/core arrangement where one polymer is surrounded by another, or may be a side-by-side arrangement, a pie arrangement, or an “islands-in-the-sea” arrangement. Conjugate fibers are taught by U.S. Pat. No. 5,108,820 to Kaneko, et al., U.S. Pat. No. 4,795,668 to Krueger, et al., U.S. Pat. No. 5,540,992 to Marcher, et al., U.S. Pat. No. 5,336,552 to Strack, et al., U.S. Pat. No. 5,425,987 to Shawver, and U.S. Pat. No. 5,382,400 to Pike, et al. each being incorporated herein in their entirety by reference thereto for all purposes. For two component fibers, the polymers may be present in ratios of 75/25, 50/50, 25/75 or any other desired ratio. Additionally, polymer additives such as processing aids may be included in each zone.

As used herein, the term “machine direction” (MD) refers to the length of a fabric in the direction in which it is produced, as opposed to a “cross-machine direction” (CD) which refers to the width of a fabric in a direction generally perpendicular to the machine direction.

As used herein, the term “meltblown web” refers herein to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that are substantially continuous or discontinuous, generally smaller than 10 microns in diameter, and generally tacky when deposited onto a collecting surface.

As used herein, the term “nonwoven fabric” or “nonwoven web” refers herein to a web having a structure of individual fibers or threads which are interlaid, but not in an identifiable manner as in a knitted fabric. Nonwoven fabrics or webs have been formed from many processes such as, for example, meltblowing processes, spunbonding processes, through-air bonded carded web (also known as BCW and TABCW) processes, etc. The basis weight of nonwoven webs may generally vary, such as, from about 5, 10 or 20 gsm to about 120, 125 or 150 gsm.

As used herein, the term “spunbond web” refers herein to a web containing small diameter substantially continuous fibers. The fibers are formed by extruding a molten thermoplastic material from a plurality of fine, usually circular, capillaries of a spinneret with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are each incorporated herein in their entirety by reference thereto for all purposes. Spunbond fibers are generally not tacky when they are deposited onto a collecting surface. Spunbond fibers may sometimes have diameters less than about 40 microns, and often between about 5 to about 20 microns.

As used herein, the terms “superabsorbent polymer,” “superabsorbent” or “SAP” shall be used interchangeably and shall refer to polymers that can absorb and retain extremely large amounts of a liquid relative to their own mass. Water absorbing polymers, which are classified as hydrogels, which can be cross-linked, absorb aqueous solutions through hydrogen bonding and other polar forces with water molecules. A SAP's ability to absorb water is based in part on ionicity (a factor of the ionic concentration of the aqueous solution), and the SAP functional polar groups that have an affinity for water. SAP are typically made from the polymerization of acrylic acid blended with sodium hydroxide in the presence of an initiator to form a poly-acrylic acid sodium salt (sometimes referred to as sodium polyacrylate). Other materials are also used to make a superabsorbent polymer, such as polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. SAP may be present in absorbent articles in particle or fibrous form or as a coating on another material or fiber.

### Absorbent Article:

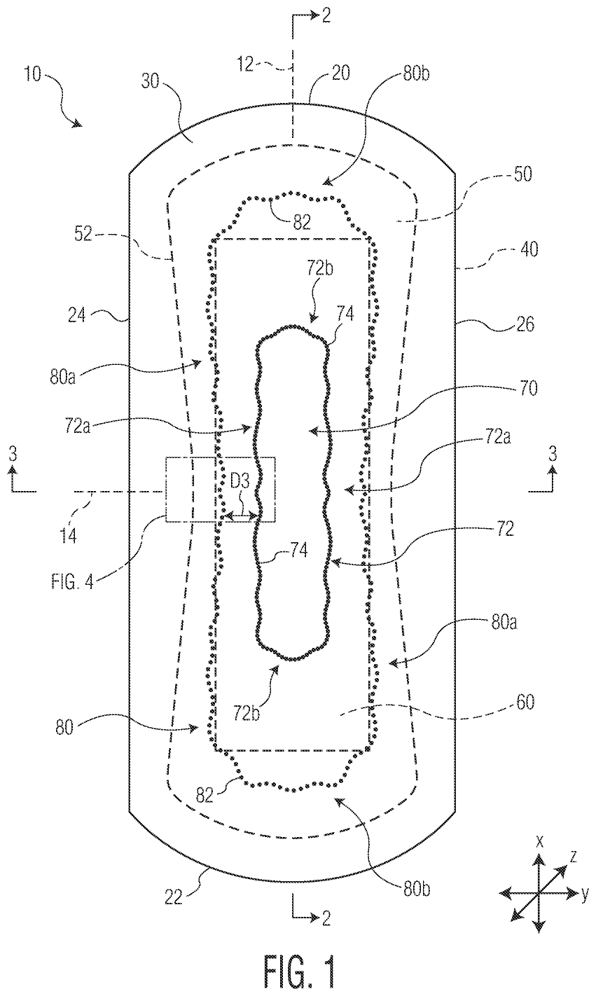

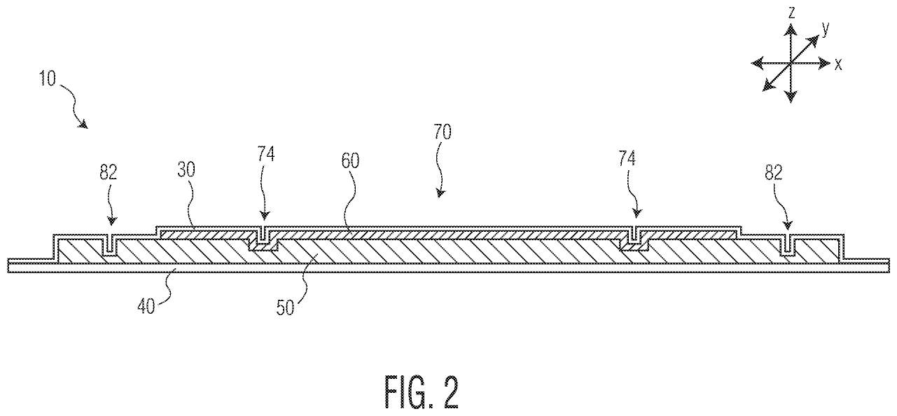

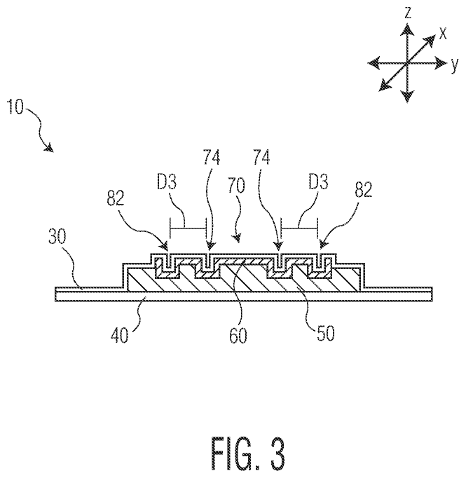

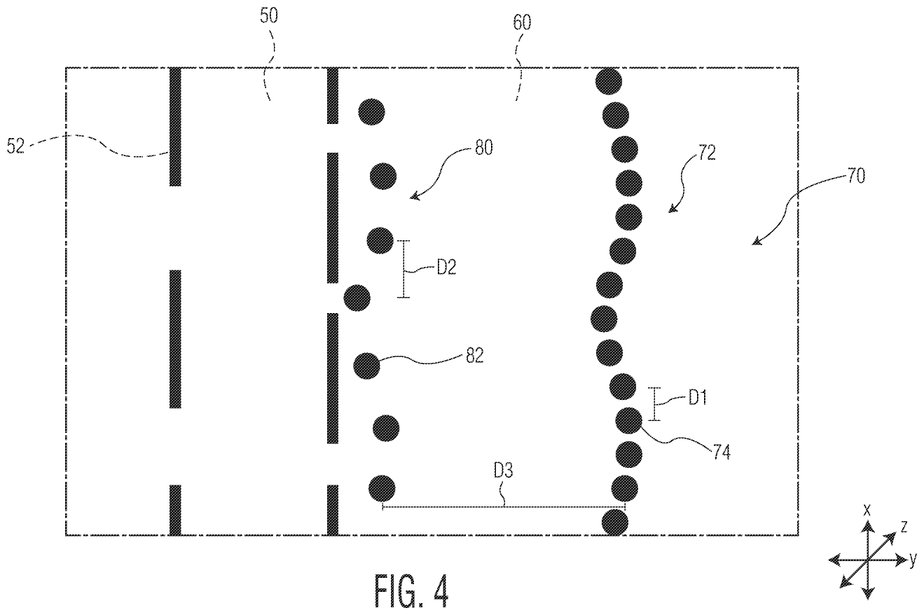

Referring to FIGS. 1-4, FIG. 1 is a top down view of an embodiment of an absorbent article 10, FIG. 2 is a cross-sectional view of the absorbent article 10 of FIG. 1 taken along line 2-2, FIG. 3 is a cross-sectional view of the absorbent article 10 of FIG. 1 taken along line 3-3, and FIG. 4 is a close-up view of a portion of the absorbent article 10 of FIG. 1. The absorbent article 10 can have a longitudinal direction (X), a transverse direction (Y), and a depth direction (Z). The absorbent article 10 can have a longitudinal direction axis 12 and a transverse direction axis 14. The absorbent article 10 can have a first transverse direction end edge 20, a second transverse direction end edge 22 opposite the first transverse direction end edge 20, and a pair of opposing longitudinal direction side edges, 24 and 26, extending between and connecting the first transverse direction end edge 20 and the second transverse direction end edge 22. In various embodiments, the absorbent article 10 can take on various geometries but will generally have a pair of opposing longitudinal direction side edges, 24 and 26, and a pair of opposing transverse direction end edges, 20 and 22. The absorbent article 10 can have a wearer facing, liquid permeable topsheet layer 30 and a garment facing, liquid impermeable backsheet layer 40. An absorbent core 50 can be positioned between the topsheet layer 30 and the backsheet layer 40. In various embodiments, a fluid intake layer 60 can be positioned between the topsheet layer 30 and the absorbent core 50.

The topsheet layer 30 and the backsheet layer 40 can both extend beyond the outermost peripheral edges of the absorbent core 50 and can be peripherally bonded together, either entirely or partially, using known bonding techniques to form a sealed peripheral edge. For example, the topsheet layer 30 and the backsheet layer 40 can be bonded together by adhesive bonding, ultrasonic bonding, or any other suitable bonding method known in the art.

The absorbent article 10 has a target acquisition region 70 which is the region of the absorbent article 10 wherein initial acquisition of body exudates from the wearer of the absorbent article 10 should occur. To identify the target acquisition region 70 for the wearer of the absorbent article 10, the target acquisition region 70 is defined by and bounded by a perimeter 72 formed of individual embossment points 74. To provide the wearer of the absorbent article 10 with the knowledge that the absorbent article 10 will be able to capture and store their body exudates, the absorbent article 10 has a second perimeter 80 formed of individual embossment points 82 positioned between the perimeter 72 defining the target acquisition region 70 and the edge 52 of the absorbent core 50.

### Topsheet Layer:

The topsheet layer 30 defines a wearer facing surface of the absorbent article 10 that may directly contact the body of the wearer and is liquid permeable to receive body exudates. The topsheet layer 30 is desirably provided for comfort and conformability and functions to direct body exudates away from the body of the wearer, through its own structure, and towards the absorbent core 50. The topsheet layer 30 desirably retains little to no liquid in its structure, so that it provides a relatively comfortable and non-irritating surface next to the skin of the wearer of the absorbent article 10.

The topsheet layer 30 can be a single layer of material, or alternatively, can be multiple layers that have been laminated together. The topsheet layer 30 can be constructed of any material such as one or more woven sheets, one or more fibrous nonwoven sheets, one or more film sheets, such as blown or extruded films, which may themselves be of single or multiple layers, one or more foam sheets, such as reticulated, open cell or closed cell foams, a coated nonwoven sheet, or a combination of any of these materials. Such combination can be adhesively, thermally, or ultrasonically laminated into a unified planar sheet structure to form a topsheet layer 30.

In various embodiments, the topsheet layer 30 can be constructed from various nonwoven webs such as meltblown webs, spunbond webs, spunlace webs, hydroentangled spunlace webs, or through air bonded carded webs. Examples of suitable topsheet layer 30 materials can include, but are not limited to, natural fiber webs (such as cotton), rayon, hydroentangled webs, bonded carded webs of polyester, polypropylene, polyethylene, nylon, or other heat-bondable fibers (such as bicomponent fibers), polyolefins, copolymers of polypropylene and polyethylene, linear low-density polyethylene, and aliphatic esters such as polylactic acid. Finely perforated films and net materials can also be used, as can laminates of/or combinations of these materials. An example of a suitable topsheet layer 30 is a material that includes natural fibers. An example of a suitable topsheet layer 30 can be a bonded carded web made of polypropylene and polyethylene such as that obtainable from Sandler Corporation, Germany. U.S. Pat. No. 4,801,494 to Datta, et al., and U.S. Pat. No. 4,908,026 to Sukiennik, et al., and WO 2009/062998 to Texol teach various other topsheet materials that may be used as the topsheet layer 30, each of which is hereby incorporated by reference thereto in its entirety. Additional topsheet layer 30 materials can include, but are not limited to, those described in U.S. Pat. No. 4,397,644 to Matthews, et al., U.S. Pat. No. 4,629,643 to Curro, et al., U.S. Pat. No. 5,188,625 to Van Iten, et al., U.S. Pat. No. 5,382,400 to Pike, et al., U.S. Pat. No. 5,533,991 to Kirby, et al., U.S. Pat. No. 6,410,823 to Daley, et al., and U.S. Publication No. 2012/0289917 to Abuto, et al., each of which is hereby incorporated by reference thereto in its entirety.

In various embodiments, the topsheet layer 30 may contain a plurality of apertures formed therethrough to permit body exudates to pass more readily into the absorbent article 10. The apertures may be randomly or uniformly arranged throughout the topsheet layer 30. The size, shape, diameter, and number of apertures may be varied to suit an absorbent article's 10 particular needs.

In various embodiments, the topsheet layer 30 can have a basis weight ranging from about 5, 10, 15, 20, 25, or 30 gsm to about 50, 100, 120, 125 or 150 gsm. For example, in an embodiment, a topsheet layer 30 can be constructed from a through air bonded carded web having a basis weight ranging from about 15 gsm to about 100 gsm. In another example, a topsheet layer 30 can be constructed from a through air bonded carded web having a basis weight from about 20 gsm to about 50 gsm, such as a through air bonded carded web that is readily available from nonwoven material manufacturers, such as Xiamen Yanjan Industry, Beijing, DaYuan Nonwoven Fabrics and others.

In various embodiments, the topsheet layer 30 can be at least partially hydrophilic. In various embodiments, a portion of the topsheet layer 30 can be hydrophilic and a portion of the topsheet layer 30 can be hydrophobic. In various embodiments, the portions of the topsheet layer 30 which can be hydrophobic can be either an inherently hydrophobic material or can be a material treated with a hydrophobic coating.

In various embodiments, the topsheet layer 30 can be a multicomponent topsheet layer 30 such as by having two or more different nonwoven or film materials, with the different materials placed in separate locations in the transverse direction (Y) of the absorbent article 10. For example, the topsheet layer 30 can be a two layer or multicomponent material having a central portion positioned along and straddling a longitudinal direction axis 12 of the absorbent article 10, with lateral side portions flanking and bonded to each side edge of the central portion. The central portion can be constructed from a first material and the side portions can be constructed from a material which can be the same as or different from the material of the central portion. In such embodiments, the central portion may be at least partially hydrophilic and the side portions may be inherently hydrophobic or may be treated with a hydrophobic coating. Examples of constructions of multi-component topsheet layers are generally described in U.S. Pat. No. 5,961,505 to Coe, U.S. Pat. No. 5,415,640 to Kirby, and U.S. Pat. No. 6,117,523 to Sugahara, each of which is incorporated herein by reference thereto in its entirety.

In various embodiments, a central portion of a topsheet layer 30 can be positioned symmetrically about the absorbent article 10 longitudinal direction axis 12. Such central longitudinally directed central portion can be a through air bonded carded web (“TABCW”) having a basis weight between about 15 and about 100 gsm. Previously described nonwoven, woven, and apertured film topsheet layer materials may also be used as the central portion of a topsheet layer 30. In various embodiments, the central portion can be constructed from a TABCW material having a basis weight from about 20 to about 50 gsm such as is available from Xiamen Yanjan Industry, Beijing, DaYuan Nonwoven Fabrics, and others. Alternatively, apertured films, such as those available from such film suppliers as Texol, Italy and Tredegar, U.S.A. may be utilized. Different nonwoven, woven, or film sheet materials may be utilized as the side portions of the topsheet layer 30. The selection of such topsheet layer 30 materials can vary based upon the overall desired attributes of the topsheet layer 30. For example, it may be desired to have a hydrophilic material in the central portion and hydrophobic-barrier type materials in the side portions to prevent leakage and increase a sense of dryness in the area of the side portions. Such side portions can be adhesively, thermally, ultrasonically, or otherwise bonded to the central portion along or adjacent the longitudinally directed side edges of the central portion. Traditional absorbent article construction adhesive may be used to bond the side portions to the central portion. Either of the central portion and/or the side portions may be treated with surfactants and/or skin-health benefit agents, as are well known in the art.

Such longitudinally directed side portions can be of a single or multi-layered construction. In various embodiments, the side portions can be adhesively or otherwise bonded laminates. In various embodiments, the side portions can be constructed of an upper fibrous nonwoven layer, such as a spunbond material, laminated to a bottom layer of a hydrophobic barrier film material. Such a spunbond layer may be formed from a polyolefin, such as a polypropylene and can include a wetting agent if desired. In various embodiments, a spunbond layer can have a basis weight from about 10 or 12 gsm to about 30 or 70 gsm and can be treated with hydrophilic wetting agents. In various embodiments, a film layer may have apertures to allow fluid to permeate to lower layers, and may be either of a single layer or multi-layer construction. In various embodiments, such film can be a polyolefin, such as a polyethylene having a basis weight from about 10 to about 40 gsm. Construction adhesive can be utilized to laminate the spunbond layer to the film layer at an add-on level of between about 0.1 gsm and 15 gsm. When a film barrier layer is used in the overall topsheet layer design, it may include opacifying agents, such as film pigments, that can help the film in masking stains along the absorbent article 10 side edges, thereby serving as a masking element. In such a fashion, the film layer can serve to limit visualization of a fluid insult stain along the absorbent article 10 side edges when viewed from above the topsheet layer 30. The film layer may also serve as a barrier layer to prevent rewet of the topsheet layer 30 as well as to prevent the flow of fluid off the side edges of the absorbent article 10. In various embodiments, the side portions can be laminates such as a spunbond-meltblown-meltblown-spunbond layer (“SMMS”) laminate, spunbond-film laminate, or alternatively, other nonwoven laminate combinations.

### Backsheet Layer:

The backsheet layer 40 is generally liquid impermeable and is the portion of the absorbent article 10 which faces the garment of the wearer. The backsheet layer 40 can permit the passage of air or vapor out of the absorbent article 10 while still blocking the passage of liquids. Any liquid impermeable material may generally be utilized to form the backsheet layer 40. The backsheet layer 40 can be composed of a single layer or multiple layers, and these one or more layers can themselves comprise similar or different materials. Suitable material that may be utilized can be a microporous polymeric film, such as a polyolefin film of polyethylene or polypropylene, nonwovens and nonwoven laminates, and film/nonwoven laminates. The particular structure and composition of the backsheet layer 40 can be selected from various known films and/or fabrics with the particular material being selected as appropriate to provide the desired level of liquid barrier, strength, abrasion resistance, tactile properties, aesthetics and so forth. In various embodiments, a polyethylene film can be utilized that can have a thickness in the range of from about 0.2 or 0.5 mils to about 3.0 or 5.0 mils. An example of a backsheet layer 40 can be a polyethylene film such as that obtainable from Pliant Corporation, Schaumburg, IL, USA. Another example can include calcium carbonate-filled polypropylene film. In still another embodiment, the backsheet layer 40 can be a hydrophobic nonwoven material with water barrier properties such as a nonwoven laminate, an example of which can be a spunbond, meltblown, meltblown, spunbond, four-layered laminate. The backsheet layer 40 can, therefore, be of a single or multiple layer construction, such as of multiple film layers or laminates of film and nonwoven fibrous layers. Suitable backsheet layers 40 can be constructed from materials such as those described in U.S. Pat. No. 4,578,069 to Whitehead, et al., U.S. Pat. No. 4,376,799 to Tusim, et al., U.S. Pat. No. 5,695,849 to Shawver, et al., U.S. Pat. No. 6,075,179 to McCormack, et al., and U.S. Pat. No. 6,376,095 to Cheung, et al., each of which are hereby incorporated by reference thereto in its entirety.

### Absorbent Core:

An absorbent core 50 can be positioned between the topsheet layer 30 and the backsheet layer 40 of the absorbent article 10. The absorbent core 50 can generally be any single layer structure or combination of layer components, which can demonstrate some level of compressibility, conformability, be non-irritating to the wearer's skin, and capable of absorbing and retaining liquids and other body exudates. In various embodiments, the absorbent core 50 can be formed from a variety of different materials and can contain any number of desired layers. For example, the absorbent core 50 can include one or more layers (e.g., two layers) of absorbent web material of cellulosic fibers (e.g., wood pulp fibers), other natural fibers, synthetic fibers, woven or nonwoven sheets, scrim netting, or other stabilizing structures, superabsorbent material, binder materials, surfactants, selected hydrophobic and hydrophilic materials, pigments, lotions, odor control agents or the like, as well as combinations thereof. In an embodiment, the absorbent web material can include a matrix of cellulosic fluff and can also include superabsorbent material. The cellulosic fluff can comprise a blend of wood pulp fluff. An example of wood pulp fluff can be identified with the trade designation NB416, available from Weyerhaeuser Corp., and is a bleached, highly absorbent wood pulp containing primarily soft wood fibers.

In various embodiments, if desired, the absorbent core 50 can include an optional amount of superabsorbent material. Examples of suitable superabsorbent material can include poly(acrylic acid), poly(methacrylic acid), poly(acrylamide), poly(vinyl ether), maleic anhydride copolymers with vinyl ethers and α-olefins, poly(vinyl pyrrolidone), poly(vinylmorpholinone), poly(vinyl alcohol), and salts and copolymers thereof. Other superabsorbent materials can include unmodified natural polymers and modified natural polymers, such as hydrolyzed acrylonitrile-grafted starch, acrylic acid grafted starch, methyl cellulose, chitosan, carboxymethyl cellulose, hydroxypropyl cellulose, and natural gums, such as alginates, xanthan gum, locust bean gum, and so forth. Mixtures of natural and wholly or partially synthetic superabsorbent polymers can also be useful. The superabsorbent material can be present in the absorbent core 50 in any amount as desired.

Regardless of the combination of absorbent materials used in the absorbent core 50, the absorbent materials can be formed into a web structure by employing various conventional methods and techniques. For example, the absorbent web can be formed by techniques such as, but not limited to, a dry-forming technique, an air forming technique, a wet forming technique, a bonded carded forming technique, a foam forming technique, or the like, as well as combinations thereof. A coform nonwoven material can also be employed. Methods and apparatus for carrying out such techniques are well known in the art.

The shape of the absorbent core 50 can vary as desired and can comprise any one of various shapes including, but not limited to, triangular, rectangular, dog-bone, elliptical, trapezoidal, T-shape, I-shape, and hourglass shapes. In various embodiments, the absorbent core 50 can have a shape that generally corresponds with the overall shape of the absorbent article 10. The dimensions of the absorbent core 50 can be substantially similar to those of the absorbent article 10, however, it will be appreciated that the dimensions of the absorbent core 50 while similar, will often be less than those of the overall absorbent article 10, in order to be adequately contained therein. The size and the absorbent capacity of the absorbent core 50 should be compatible with the size of the intended wearer and the liquid loading imparted by the intended use of the absorbent article 10.

By way of example, suitable materials and/or structures for the absorbent core 50 can include, but are not limited to, those described in U.S. Pat. No. 4,610,678 to Weisman, et al., U.S. Pat. No. 6,060,636 to Yahiaoui, et al., U.S. Pat. No. 6,610,903 to Latimer, et al., U.S. Pat. No. 7,358,282 to Krueger, et al., and U.S. Publication No. 2010/0174260 to Di Luccio, et al. each of which is hereby incorporated by reference thereto in its entirety.

In various embodiments, an absorbent core 50 can be a single layer structure and can include, for example, a matrix of cellulosic fluff and superabsorbent material. In various embodiments, an absorbent core 50 can have at least two layers of material, such as, for example, a body facing layer and a garment facing layer. In various embodiments, the two layers can be identical to each other. In various embodiments, the two layers can be different from each other. In such embodiments, the two layers can provide the absorbent article 10 with different absorption properties as deemed suitable. In various embodiments, the body facing layer of the absorbent core 50 may be constructed of an airlaid material and the garment facing layer of the absorbent core 50 may be constructed of a superabsorbent polymer-containing compressed sheet. In such embodiments, the airlaid material can have a basis weight from about 40 to about 200 gsm and the superabsorbent polymer-containing compressed sheet can be a cellulosic fluff based material that can be a combination of cellulosic pulp and SAP enclosed with a tissue carrier and having a basis weight from about 40 to about 400 gsm. In various embodiments, the body facing layer of the absorbent body 10 may be constructed of a through-air bonded carded web material and the garment facing layer of the absorbent body 10 may be constructed of a matrix of cellulosic fluff sheet. In various embodiments, the body facing layer of the absorbent body 10 may be constructed of a through-air bonded carded web material and the garment facing layer of the absorbent body 10 may be constructed of an airlaid material.

### Fluid Intake Layer:

In various embodiments, the absorbent article 10 can include a liquid permeable fluid intake layer 60 positioned between the topsheet layer 30 and the absorbent core 50. Such a fluid intake layer 60 can be made of a material that can be capable of rapidly transferring, in the depth direction (Z), body exudates that are delivered to the topsheet layer 30. The fluid intake layer 60 can generally have any shape and/or size desired.

Any of a variety of different materials can be capable of being used for the fluid intake layer 60 to accomplish the above-mentioned functions. The material may be synthetic, cellulosic, or a combination of synthetic and cellulosic materials. The fluid intake layer 60 can be constructed from any woven or nonwoven material. For example, the fluid intake layer 60 can be constructed as an airlaid or TABCW material. For example, airlaid cellulosic tissues may be suitable for use in the fluid intake layer 60. The airlaid cellulosic tissue may have a basis weight ranging from about 10 or 100 gsm to about 250 or 300 gsm. The airlaid cellulosic tissue can be formed from hardwood and/or softwood fibers. An airlaid cellulosic tissue can have a fine pore structure and can provide an excellent wicking capacity, especially for menses.

### Embossing:

Referring to FIGS. 1-4, the absorbent article 10 has a target acquisition region 70 which is the region of the absorbent article 10 wherein initial acquisition of body exudates from the wearer of the absorbent article 10 should occur. To identify the target acquisition region 70 for the wearer of the absorbent article 10, the target acquisition region 70 is defined by and bounded by a perimeter 72 formed of individual embossment points 74. To provide the wearer of the absorbent article 10 with the knowledge that the absorbent article 10 will be able to capture and store their body exudates, the absorbent article 10 has a second perimeter 80 formed of individual embossment points 82 positioned between the perimeter 72 defining the target acquisition region 70 and the edge 52 of the absorbent core 50.

In various embodiments, depending upon the overall structure of the absorbent article 10, each individual embossment point, 74 and 82, of each perimeter, 72 and 80, respectively, can cause a deformation in one or more layers of the absorbent article 10. For example, an individual embossment point, 74 or 82, may cause a deformation in the topsheet layer 30 and the absorbent core 50. As another example, an individual embossment point, 74 or 82, may cause a deformation in the topsheet layer 30, the fluid intake layer 60, and the absorbent core 50. Each embossment point, 74 and 82, can have any shape as desired such as, for example, circle, oval, square, triangle, diamond, rectangle. Each embossment point, 74 and 82, can have a size dimension to provide the overall desired level of flexibility to the absorbent article 10. In various embodiments, each embossment point, 74 and 82, can have a length in the longitudinal direction (X) of about 1 mm. In various embodiments, each embossment point, 74 and 82, can have a width in the transverse direction (Y) of about 1 mm. In various embodiments, each embossment point, 74 and 82, can have a length in the longitudinal direction (X) of about 1 mm and a width in the transverse direction (Y) of about 1 mm.

Each embossment point, 74 and 82, can be incorporated into the absorbent article 10 by utilizing embossing pins wherein each embossing pin has the shape desired for each embossment point, 74 and 82. For example, an embossing pin can have a round shape which can produce an embossment point, 74 or 82, having a circular shape. An example of a process to form each perimeter, 72 and 80, can include thermal bonding wherein the absorbent article 10 is passed between a pair of rollers (e.g. steel, rubber, etc.) where one of the rollers has the embossing pins (e.g. an embossing roller) extending outwardly from its surface and the other roller is flat (e.g. a smooth roller). One or both of the rollers can be heated. Passing the absorbent article 10 between such a pair of rollers can result in the embossing pins impressing the absorbent article 10 with the desired pattern of embossment points corresponding to the pattern of embossing pins.

The perimeter 72 of embossment points 74 defining the target acquisition region 70 can be formed in any suitable pattern to not only define the target acquisition region 70 for the wearer of the absorbent article 10, but also to create an aesthetically pleasing surface to the absorbent article 10. The perimeter 72 of embossment points 74 can be provided in either a symmetric or asymmetric manner to the absorbent article 10. As an example, as illustrated in FIG. 1-3, the perimeter 72 of embossment points 74 is a generally symmetrical oblong shape with each individual embossment point 74 arranged in a spatial relationship with each adjacent individual embossment point 74 to provide an overall curvilinear pattern to the generally oblong shape. As shown in the exemplary illustrations of FIGS. 1-3, the individual embossment points 74 of the perimeter 72 form an opposing pair of longitudinally extending side portions 72a which extend in the longitudinal direction (X) of the absorbent article 10. The individual embossment points 74 of the perimeter 72 also form an opposing pair of transversely extending end portions 72b which connect the longitudinally extending side portions 72a. The perimeter 72 of individual embossment points 74 defining the target acquisition region 70 can be any size and shape as desired and as deemed suitable to communicate to the wearer of the absorbent article 10 the location of the target acquisition region 70 of the absorbent article 10. Referring to FIG. 4, a close-up of a portion of the absorbent article 10 of FIG. 1, in various embodiments, the individual embossment points 74 of the perimeter 72 can be spaced apart from each other by a distance D1, as measured as the distance from the center of one embossment point 74 to the center of the adjacent embossment point 74 (i.e., a center-to-center distance), from 0.7, 0.8, 0.9, 1, 1.1, 1.2, 1.3, 1.4, 1.5, 1.6, 1.7, or 1.8 mm to 1.9, 2, 2.1, 2.2, 2.3, 2.4, 2.5, 2.6, 2.7, 2.8, 2.9, 3, 3.1, 3.2, 3.3, 3.4, or 3.5 mm.

The perimeter 80 of embossment points 82 positioned between the perimeter 72 defining the target acquisition region 70 and the edge 52 of the absorbent core 50 can be formed in any suitable pattern to provide knowledge to the wearer of the absorbent article 10 that the absorbent article 10 will capture and contain their body exudates, but also to create an aesthetically pleasing surface to the absorbent article 10. The perimeter 80 of embossment points 82 can be provided in either a symmetric or asymmetric manner to the absorbent article 10. As an example, as illustrated in the FIGS. 1-3, the perimeter 80 of embossment points 82 is a generally symmetrical oblong shape with each individual embossment point 82 arranged in a spatial relationship with each adjacent individual embossment point 82 to provide an overall curvilinear pattern to the generally oblong shape. As shown in the exemplary illustrations, the individual embossment points 82 of the perimeter 80 form an opposing pair of longitudinally extending side portions 80a which extend the longitudinal length of the absorbent article 10. The embossment points 82 of the perimeter 80 also form an opposing pair of transversely extending end portions 80b which connect the longitudinally extending side portions 80a. The perimeter 80 of individual embossment points 82 can be any size and shape as desired to assure the wearer of the absorbent article 10 that the absorbent article 10 will capture and contain their body exudates. Referring to FIG. 4, a close-up of a portion of the absorbent article 10, in various embodiments, the individual embossment points 82 of the perimeter 80 can be spaced apart from each other by a distance D2, as measured as the distance from the center of one embossment point 82 to the center of the adjacent embossment point 82 (i.e., a center-to-center distance), from 0.5, 1, 1.15, 1.25, 1.5, 1.75, 2, 2.15, 2.25, 2.5, 2.75, 3, 3.15, 3.25, 3.5, or 3.75 mm to 4, 4.25, 4.5, 4.75, 5, 5.25. 5.5, 5.75, 6, or 6.25 mm.

The spatial relationship between the perimeter 72 of individual embossment points 74 and the perimeter 80 of individual embossment points 82 can impact the fit properties of the absorbent article 10. The flexibility, compressibility, and ability of the absorbent article 10 to move and shift with the wearer of the absorbent article 10 can be negatively impacted if the spatial relationship between the perimeter 72 of individual embossment points 74 and the perimeter 80 of individual embossment points 82 is too close. This is of particular concern in the portion of the absorbent article 10 containing the target acquisition region 70. Failure of an absorbent article 10 to be flexible and to move and shift with the wearer of the absorbent article 10 can result in an absorbent article 10 that is stiff and may not adequately capture body exudates from the wearer of the absorbent article 10. An absorbent article 10 which is too stiff to move and shift with the movements of the wearer, may gap away from the body of the wearer which can result in leakage of body exudates from the absorbent article 10 rather than capture and storage of body exudates in the absorbent article 10. Each embossment point, 74 and 82, compacts the fibrous material forming the absorbent core 30 and/or fluid intake layer 60 within that specific location and, therefore, increases the density of the absorbent article 10 within that specific location of the embossment point, 74 and 82. Placing the perimeter 72 of individual embossment points 74 and the perimeter 80 of individual embossment points 82 in close proximity to each other, particularly in the target acquisition region 70 of the absorbent article 10, can result in areas of the absorbent article 10 with an overall higher level of density and, therefore, a greater stiffness to the absorbent article 10, than a consumer will find comfortable. To provide an absorbent article 10 with the ability to better conform to the body of the wearer and move with the body of the wearer during usage of the absorbent article 10, an embossment point 74 in perimeter 70 is separated from an embossment point 82 in perimeter 80 by a distance D3, as measured from the center of an embossment point 74 to the center of an embossment point 82, of at least 6, 6.5, 7, 7.5, 8, 8.5, 9, 9.5, 10, or 10.5 mm. A distance D3 of at least 6 mm separating the embossment points, 74 and 82, of the two perimeters, 72 and 80, respectively, can result in an absorbent article 10 which is more flexible, compressible, and comfortable for the consumer to wear. In various embodiments, an embossment point 74 in perimeter 70 is separated from an embossment point 82 in perimeter 80 by a distance D3 of at least 10 mm.

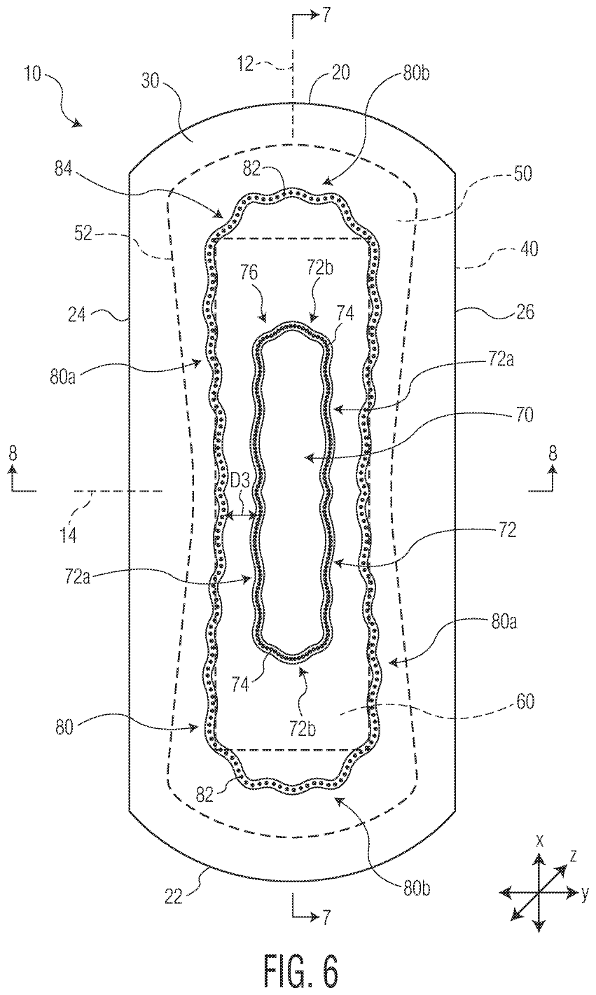

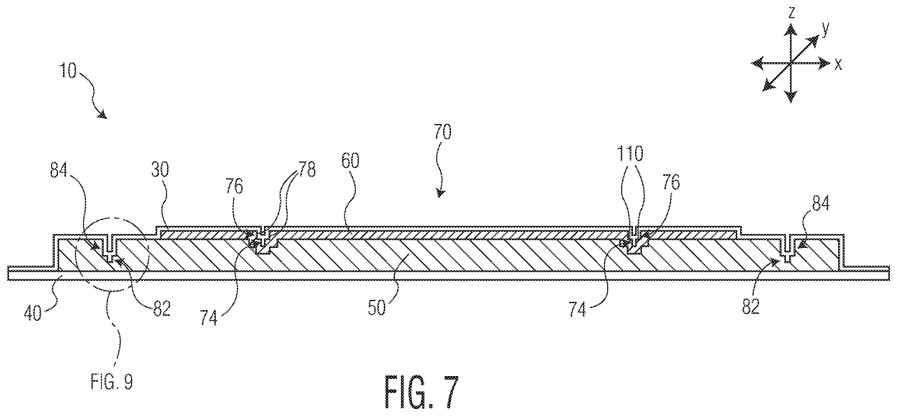

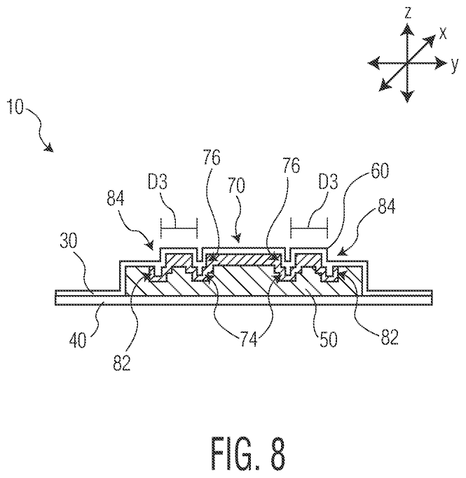

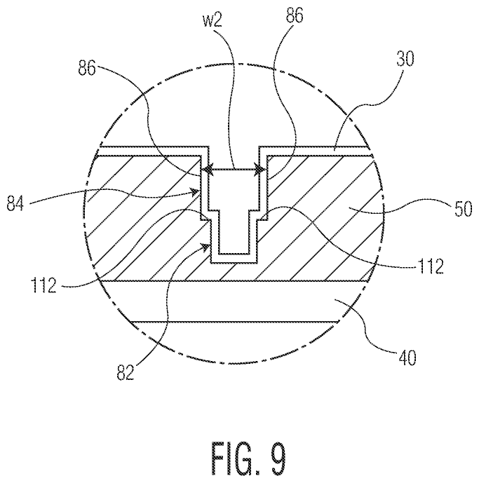

In various embodiments, to reinforce the communication to the wearer of the absorbent article 10 that the absorbent article 10 will provide the desired properties of acquisition and storage of body exudates, each of the embossment points, 74 and 82, of the perimeters, 72 and 80, respectively, may be positioned within an embossment region, 76 and 84, respectively, such as illustrated in FIGS. 6-9. FIG. 6 is a top down view of an embodiment of an absorbent article 10, FIG. 7 is a cross-sectional view of the absorbent article 10 of FIG. 6 taken along line 7-7, FIG. 8 is a cross-sectional view of the absorbent article 10 of FIG. 6 taken along line 8-8, and FIG. 9 is a close-up view of a portion of the absorbent article 10 of FIG. 7. An embossment region, 76 and 84, can provide the absorbent article 10 with additional areas of compression which are shallower in the depth direction (Z) than the compression depth, in the depth direction (Z), of an embossment point, 74 and 82, respectively. An embossment region, 76 and 84, can surround each individual embossment point, 74 and 82, respectively, and can extend between a pair of embossment points within the same perimeter. An embossment region, 76 and 84, can, therefore, enhance the definition of each perimeter, 72 and 80, respectively, within the absorbent article 10. While each embossment region, 76 and 84, can increase the density of the absorbent article 10 at the location of each embossment region, 76 and 84, as the depth of each embossment region, 76 and 84, is shallower in the depth direction (Z) than the compression depth, in the depth direction (Z), of an embossment point, 74 and 82, respectively, the material of the layers of the absorbent article 10, the absorbent core 50 and/or fluid intake layer 60, surrounding the individual embossment points, 74 and 82, can still shift and flex in response to the movements of the wearer of the absorbent article 10.

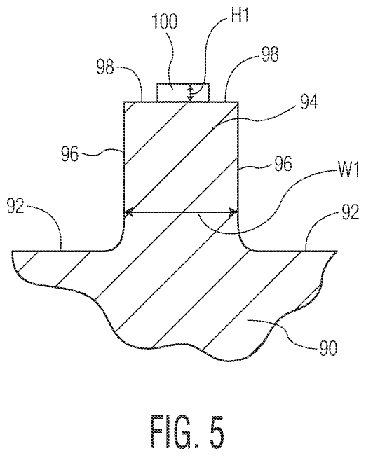

In various embodiments, an embossment region can be incorporated into an absorbent article 10 using an embossing roller. In such embodiments, embossing pins can extend from a ridge positioned on the exterior surface of the embossing roller. Referring to FIG. 5, a cross-sectional view of a portion of a ridge 94 on an embossing roller 90 is illustrated. To form an embossment region, 76 and/or 84, with individual embossment points, 74 and/or 82, respectively, positioned therein, an embossing roller 90 can have a ridge 94 extending outwardly from the surface 92 of the embossing roller 90. The ridge 94 can have a pair of sidewalls 96 and an outer surface 98. An embossing pin 100 can extend from the outer surface 98 of the ridge 94. In various embodiments, each embossing pin 100 can have any size and shape deemed suitable for the absorbent article 10. Each embossment pin 100 can have any shape as desired such as, for example, circle, oval, square, triangle, diamond, rectangle. Each embossment pin 100 can have a size dimension to provide the overall desired level of flexibility to the absorbent article 10. In various embodiments, each embossment pin 100 can have a length in the longitudinal direction (X) of about 1 mm. In various embodiments, each embossment pin 100 can have a width in the transverse direction (Y) of about 1 mm. In various embodiments, each embossment pin 100 can have a length in the longitudinal direction (X) of about 1 mm and a width in the transverse direction (Y) of about 1 mm. Each embossment pin 100 can have a height H1 that it extends outwardly away from the outer surface 98 of the ridge 94. In various embodiments, the height H1 of each embossment pin 100 can be less than 1.0 mm. In various embodiments, the height H1 of each embossment pin 100 can be from 0.4 or 0.6 mm to 0.8 or 1 mm. The ridge 94 can have a width W1 between the pair of sidewalls 96 which is wider than the width dimension of the embossing pin 100. In various embodiments, the width W1 between the pair of sidewalls 96 of the ridge 94 can be from 2 or 2.5 mm to 3 or 3.5 mm. Referring to FIGS. 6-9, utilizing embossing pins 100 on a ridge 94 on an embossing roller 90 can provide the absorbent article 10 with an embossment region, 76 and/or 84, with embossment points, 74 and/or 82, respectively, located therein. Each embossment region, 76 and 84, of the absorbent article 10 can be defined by sidewalls such as sidewalls 78 of embossment region 76 and sidewalls 86 of embossment region 84. Each embossment region, 76 and 84, can have a width W2 between the sidewalls, 78 and 86, respectively, from 2 or 2.5 mm to 3 or 3.5 mm. As the ridge 94 can have a width W1 between the pair of sidewalls 96 which is wider than the width dimension of the embossing pin 100, the portion of the outer surface 98 of the ridge 94 surrounding the embossing pin 100 can provide a transition region 110 between the sidewalls 78 of embossment region 76 and embossment point 74 in perimeter 72 as well as can provide a transition region 112 between the sidewalls 86 of embossment region 84 and the embossment point 82 in perimeter 80.

### Shaping Element:

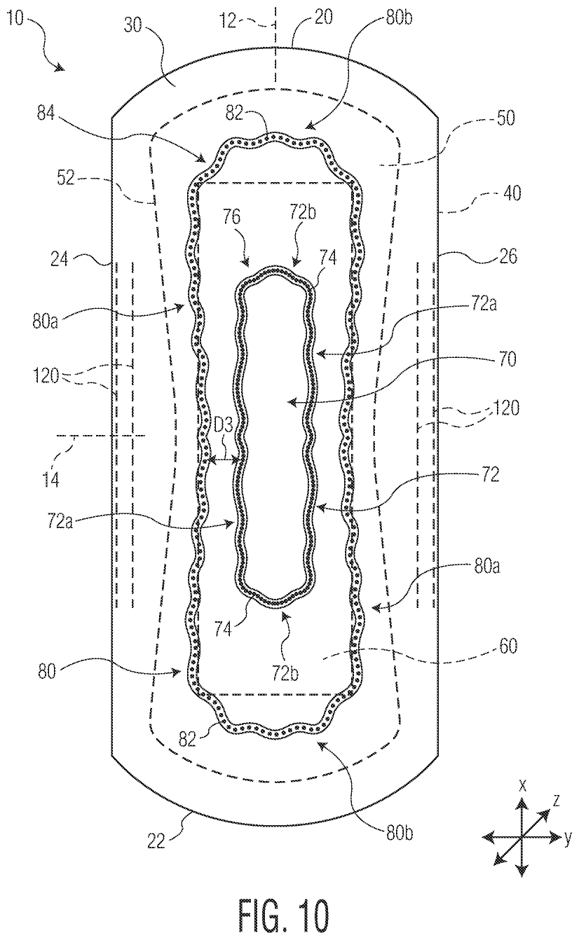

Referring to FIG. 10, in various embodiments the absorbent article 10 can further have shaping elements 120 extending in the longitudinal direction (X) of the absorbent article 10. The shaping elements 120 can be positioned between the topsheet layer 30 and the backsheet layer 40. In various embodiments, incorporation of shaping elements 120 into the absorbent article 10 can include folding the topsheet layer 30 and the backsheet layer 40 over the shaping elements 120 wherein the fold is directed downwards towards the garment facing surface of the backsheet layer 30. In various embodiments, the shaping elements 120 can be incorporated into the absorbent article 10 under tension along the longitudinal direction (X) in order to cause the absorbent article 10 to curl upwardly in the depth direction (Z). In various embodiments, the shaping element 120 is an organic elastomeric material. As used herein, an “elastomeric material” refers to a material that is able to recover its original size and shape after the removal of a deformational force. In various embodiments, an elastomeric material is a material that can be elongated greater than at least one-fourth its original dimension, and can recover towards its original dimension by at least one-tenth. In various embodiments, the shaping element 120 is an elastic material such as LYCRA® manufactured by E.I. duPont de Nemours and Company of Delaware. By way of example, suitable shaping elements and/or incorporation of shaping elements into an absorbent article can include, but are not limited to, those described in U.S. Pat. No. 10,016,311 to Loyd, et al. which is hereby incorporated by reference thereto in its entirety.

### Wings:

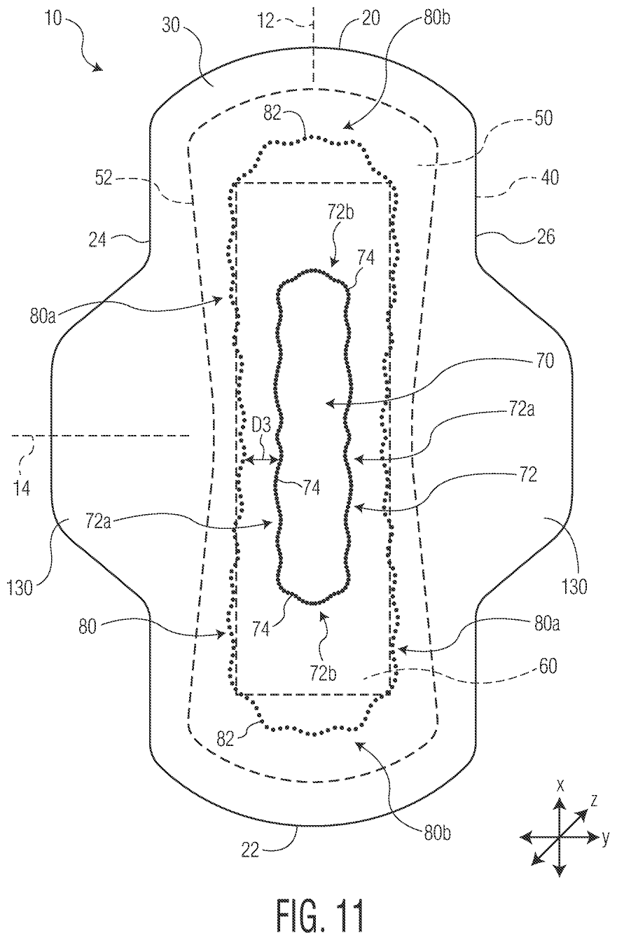

Referring to FIG. 11, in various embodiments, the absorbent article 10 can have a pair of wings 130 extending outwardly, in the transverse direction Y, from the absorbent article 10. The wings 130 can drape over the edges of the wearer's undergarment so that the wings 130 are disposed between the edges of the wearer's undergarment and her thighs. The wings 130 can serve at least two purposes. First, the wings 130 can prevent soiling of the wearer's undergarment by forming a barrier along the edges of the undergarment. Second, the wings 130 can be provided with an attachment aid, such as, for example, a garment attachment adhesive or a hook, to keep the absorbent article 10 securely and properly positioned in the undergarment. The wings 130 can wrap around the crotch region of the wearer's undergarment to aid in securing the absorbent article 10 to the wearer's undergarment when in use. Each wing 130 can fold under the crotch region of the wearer's undergarment and the attachment aid can either form a secure attachment to the opposite wing 130 or directly to the surface of the wearer's undergarment. In various embodiments, the wings 130 can be an extension of materials forming the topsheet layer 30 and/or the backsheet layer 40, such that the wings 130 can be of a unitary construction with the absorbent article 10. In various embodiments, the wings 130 can be constructed of materials similar to the topsheet layer 30, the backsheet layer 40 or combinations of these materials. In various embodiments, the wings 130 can be separate elements bonded to the main body of the absorbent article 10. It is to be understood that the wings 130 are optional and, in various embodiments, an absorbent article 10 can be configured without wings 130.

### Example

To evaluate the compressibility of an absorbent article 10 wherein the distance D3 between the perimeter 72 and the perimeter 80 is close versus where the two perimeters, 72 and 80, are spaced further apart from each other, three different absorbent articles were tested for side compressibility. Each of the Control, Code 1, and Code 2 samples had the following material structure:
Topsheet Layer—Cotton spunlace liner, 30 g/m²
Backsheet Layer—Polyethylene film, 25 g/m²
Absorbent Core—Hourglass shape, Fluff Cellulose Pulp, 280 g/m², and Sodium Polyacrylate, 370 g/m², wrapped in cellulose tissue, 18 g/m²
Fluid Intake Layer—Rectangular shape, Through Air Bonded Carded Web of Bicomponent Fiber, 42 g/m²
Shaping Elements—Invista spooled Lycra HyFit Fiber, 470 dtex

Code 1 and Code 2 had the same absorbent article shape as depicted in FIG. 10 with each of the absorbent core and fluid intake layer having the same shapes as depicted in FIG. 10. The total pad length in the longitudinal direction (X) between each of the transverse direction end edges, 20 and 22, was 256 mm and the total width in the transverse direction (Y) between the longitudinal direction side edges, 24 and 26, was 105 mm. The absorbent core had an hourglass shape wherein the greatest width, in the transverse direction (Y), at the widest portions of the absorbent core was 80 mm and the narrowest width of the absorbent core, in the transverse direction (Y), at the intersection of the longitudinal axis and the transverse axis was 64 mm. The fluid intake layer had a rectangular shape and a total length, in the longitudinal direction (X), of 168 mm and a total width, in the transverse direction (Y), of 49 mm. The absorbent articles of each of Code 1 and Code 2 were symmetrical about each of the longitudinal axis and the transverse axis. The absorbent core and the fluid intake layer were each symmetrical about each of the longitudinal axis and the transverse axis. The shaping elements were two strands of Lycra fiber, each strand having a total longitudinal direction (X) stretched length of 115 mm, and spaced apart from each other by 5 mm. A distance of 1 mm separated each longitudinal direction side edge from the closest shaping element. The shaping elements were symmetrically arranged about each of the longitudinal axis and transverse axis.

Code 1 had a perimeter 72 having embossment points 74 positioned within an embossment region 76 defining a target acquisition region 70. The perimeter 72 shape and configuration were as illustrated in FIG. 10. The embossment points 74 of perimeter 72 were in the shape of circles with a diameter of 1 mm and were spaced apart from each other by a center-to-center distance of 3 mm. The embossment region 76 had a width W2 between the sidewalls 78 of the embossment region 76 of 2 mm. Code 1 had a perimeter 80 having embossment points 82 positioned within an embossment region 84. The perimeter 80 shape and configuration were as illustrated in FIG. 10. The embossment points 82 of perimeter 80 were in the shape of circles with a diameter of 1 mm and were spaced apart from each other by a center-to-center distance of 6 mm. The embossment region 84 had a width W2 between the sidewalls 86 of the embossment region 84 of 2 mm. The closest distance D3 between the center of an embossment point 74 in the perimeter 72 and the center of an embossment point 82 in the perimeter 80 was 10.7 mm.

Code 2 had a perimeter 72 having embossment points 74 positioned within an embossment region 76 defining a target acquisition region 70. The perimeter 72 shape and configuration were as illustrated in FIG. 10. The embossment points 74 of perimeter 72 were in the shape of circles with a diameter of 1 mm and were spaced apart from each other by a center-to-center distance of 1.5 mm. The embossment region 76 had a width W2 between the sidewalls 78 of the embossment region 76 of 3.4 mm. Code 2 had a perimeter 80 having embossment points 82 positioned within an embossment region 84. The perimeter 80 shape and configuration were as illustrated in FIG. 10. The embossment points 82 of perimeter 80 were in the shape of circles with a diameter of 1 mm and were spaced apart from each other by a center-to-center distance of 1.3 mm. The embossment region 84 had a width W2 between the sidewalls 86 of the embossment region 84 of 3.4 mm. The closest distance D3 between the center of an embossment point 74 in the perimeter 72 and the center of an embossment point in the perimeter 80 is 5.6 mm.

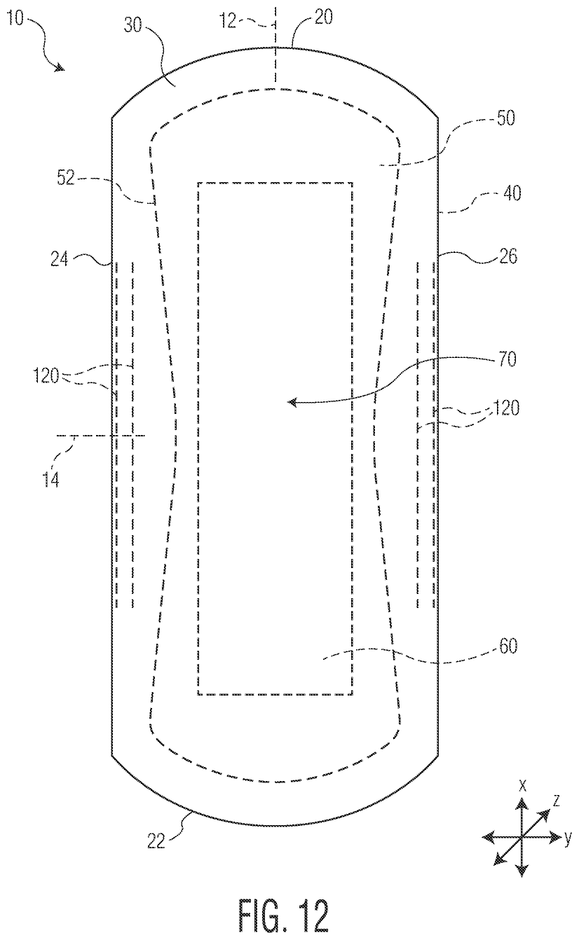

The Control sample had no perimeters of individual embossment points within embossment regions. The Control had the same absorbent article shape as depicted in FIG. 12 with each of the absorbent core and fluid intake layer having the same shapes as depicted in FIG. 12. The total pad length in the longitudinal direction (X) between each of the transverse direction end edges, 20 and 22, was 256 mm and the total width in the transverse direction (Y) between the longitudinal direction side edges, 24 and 26, was 105 mm. The absorbent core had an hourglass shape wherein the greatest width, in the transverse direction (Y), at the widest portions of the absorbent core was 80 mm and the narrowest width of the absorbent core, in the transverse direction (Y), at the intersection of the longitudinal axis and the transverse axis was 64 mm. The fluid intake layer had a rectangular shape and a total length, in the longitudinal direction (X), of 168 mm and a total width, in the transverse direction (Y), of 49 mm. The absorbent article of the Control was symmetrical about each of the longitudinal axis and the transverse axis. The absorbent core and the fluid intake layer were each symmetrical about each of the longitudinal axis and the transverse axis. The shaping elements were two strands of Lycra fiber, each strand having a total longitudinal direction (X) stretched length of 115 mm, and spaced apart from each other by 5 mm. A distance of 1 mm separated each longitudinal direction side edge from the closest shaping element. The shaping elements were symmetrically arranged about each of the longitudinal axis and transverse axis.

The thickness of each of Code 1, Code 2, and the Control were 5 mm. Thickness of each of Code 1, Code 2, and the Control was measured at the center of each absorbent article using a Mitutoyo digimatic indicator (543-562A), following removal of the shaping elements.

The Control, Code 1, and Code 2 absorbent article samples were tested for side compressibility. Six samples of each of the absorbent articles for the Control, Code 1, and Code 2 were tested for side compressibility on a MTS Criterian 42 tensile tester using Test Macro, for Testworks 4 software. The tensile tester grips were Instron part #2712-041 and the tensile tester rubber faces were Instron part #2702-309 (size 76×25 mm). The load cell was 0.62″ Dia. Bore×M-6 metric threaded stud. The tensile tester was warmed up per the manufacturer's manual and calibrated with the test conditions as follows:
Crosshead Speed=127±5 mm per minute
Gauge Length=Absorbent Core width minus 8 mm
End Compression Distance=30±1 mm
Load Unit=Grams-force
Load Limit Hi=Set to match the load cell capacity

Each test sample was conditioned at 23±2° C. and 50±5% relative humidity for at least 2 hours. The shaping elements were removed from each test sample. The entire sample was measured and the center of the sample marked. The center of the absorbent core of each sample was marked and then 38 mm to the left and right, in the longitudinal direction, of the center of the absorbent core was marked for a total distance of 76 mm between the two marks. The narrowest point, in the transverse direction, of the absorbent core between the two markings indicating the 76 mm was located and measured. At the location of the narrowest point, in the transverse direction, a spot was marked 4 mm from each longitudinal direction side edge of the absorbent core. The 4 mm spacing marked on each side of the absorbent core is the placement of the grips utilized in the tensile tester. The grip spacing was verified by adjusting the crosshead distance between the grips and editing the Gauge Length setting in the test program for the specific absorbent core width. Each test sample was placed into the tensile tester by first inserting one side of the sample at the marked located which is 4 mm into the absorbent core into the upper grip of the tensile tester. The test sample was then allowed to drape naturally into the opening of the bottom grip of the tensile tester and the grip was then closed. The load channel was zeroed and the crosshead was started allowed the sample to fold naturally. At the conclusion of the test, the sample was removed from the tensile tester.

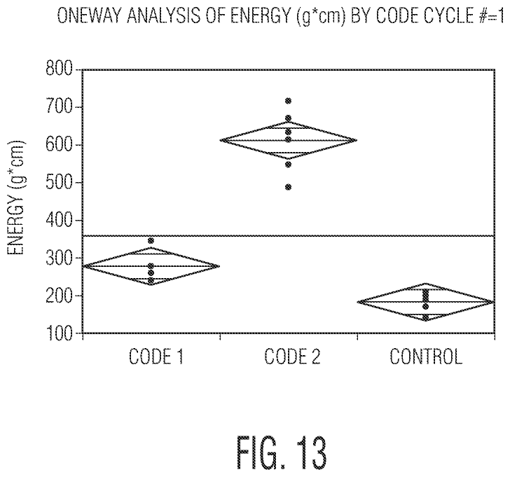

The results of this testing can be seen in FIG. 13 and Table 1 below.

TABLE 1
Means for Oneway Anova

| | | | | Lower | Upper |
|---|---|---|---|---|---|
| | Number | Mean | Standard | 95% | 95% |
| Sample | Tested | (g*cm) | Error* | (g*cm) | (g*cm) |
| Control | 6 | 180.533 | 22.212 | 133.19 | 227.88 |
| Code 1 | 6 | 275.200 | 22.212 | 227.86 | 322.54 |
| Code 2 | 6 | 611.400 | 22.212 | 564.06 | 658.74 |

*Standard Error uses a pooled estimate of error variance

As can be seen in FIG. 13 and Table 1, the Code 1 absorbent article required approximately 50% more energy to compress than the Control absorbent article, while the Code 2 absorbent article required greater than three times as much energy to compress than the Control absorbent article. Thus, Code 2 wherein the distance separating an embossing point 74 of perimeter 72 from an embossing point 82 of perimeter 80 is less than 6 mm is more stiff, less flexible, and less compressible than the Code 1 absorbent article.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a. a topsheet layer and a backsheet layer;
b. an absorbent core comprising an absorbent core edge wherein the absorbent core is positioned between the topsheet layer and the backsheet layer;
c. a target acquisition region bounded by a first perimeter comprising a first plurality of embossment points; and
d. a second perimeter comprising a second plurality of embossment points positioned between the first perimeter and the absorbent core edge;
wherein a center of each of the first plurality of embossment points is separated by a first distance from a center of each of the second plurality of embossment points and the first distance is at least 6 mm.

2. The absorbent article of claim 1 wherein the first distance is at least 10 mm.

3. The absorbent article of claim 1 wherein the first plurality of embossment points is positioned within a first embossment region.

4. The absorbent article of claim 3 wherein the first embossment region has a pair of sidewalls separated from each other by a distance of 2 to 3.5 mm.

5. The absorbent article of claim 1 wherein the second plurality of embossment points is positioned within a second embossment region.

6. The absorbent article of claim 5 wherein the second embossment region has a pair of sidewalls separated from each other by a distance of 2 to 3.5 mm.

7. The absorbent article of claim 1 wherein each of the first embossment points within the first plurality of embossment points are separated from each other by a second distance of from 0.7 mm to 3.5 mm.

8. The absorbent article of claim 1 wherein each of the second embossment points within the plurality of second embossment points are separated from each other by a third distance of from 0.5 mm to 6.25 mm.

9. The absorbent article of claim 1 further comprising a fluid intake layer positioned between the absorbent core and the fluid intake layer.

10. The absorbent article of claim 1 further comprising a shaping element.

11. The absorbent article of claim 1 further comprising a pair of wings.
